(19) United States
(12) Patent Application Publication
(10) Pub. No.: US 2008/0269339 A1
(43) Pub. Date: Oct. 30, 2008

(54) COMBINED USE OF EGF PATHWAY INHIBITORS AND DIFFERENTIATION PROMOTING COMPOUNDS

(76) Inventors: Thomas Robert Sutter, Memphis, TN (US); Carrie Hayes Sutter, Memphis, TN (US)

Correspondence Address: BUTLER, SNOW, O'MARA, STEVENS & CANNADA PLLC, 6075 POPLAR AVENUE, SUITE 500, MEMPHIS, TN, 38119, US

(21) Appl. No.: 12/110,085

(22) Filed: Apr. 25, 2008

Related U.S. Application Data
(60) Provisional application No. 60/926,249, filed on Apr. 26, 2007.

(51) Int. Cl. A61K 31/19; A61K 31/045; A61K 31/164; A61P 29/00

(52) U.S. Cl. 514/578; 514/729; 514/613

(57) ABSTRACT

The present invention is a pharmaceutical composition and method for its use in the treatment of chronic inflammatory skin diseases such as psoriasis. The pharmaceutical composition combines an inhibitor of the EGF receptor signaling pathway with an agent that promotes cell differentiation. This pharmaceutical results in superior treatment of chronic inflammatory skin diseases than methods presently in use.

| | |
|---|---|
| TITLE OF THE INVENTION: | Combined Use of EGF Pathway |
| | Inhibitors and Differentiation |
| | Promoting Compounds |
| INVENTORS: | Thomas Robert Sutter |
| | Carrie Hayes Sutter |
| CROSS REFERENCE RELATED | This application claims the |
| TO PATENT APPLICATION: | benefit of U.S. Ser. No. |
| | 60/926,249 filed Apr. 26, 2007 |
| | under 35 U.S.C. § 1.119(e) |
| | (hereby specifically incorporated |
| | by reference in its entirety) |

### FIELD OF THE INVENTION

The present invention relates generally to a treatment for chronic inflammatory skin diseases, including but not limited to psoriasis, and more specifically to a novel therapeutic compound and method for its use in the treatment of these diseases.

### BACKGROUND OF THE INVENTION

Chronic inflammatory skin diseases are extremely prevalent and include diseases such as psoriasis. More than 5 million Americans have psoriasis, and they spend between $1.6 billion and $3.2 billion each year to treat the disease, according to the National Psoriasis Foundation (NPF). Between 150,000 and 260,000 new cases are diagnosed each year, including 20,000 in children younger than 10. It occurs in all age groups and about equally in men and women. People with psoriasis may suffer discomfort, restricted motion of joints, and emotional distress. This disease is common, chronic, and costly, both in monetary terms and in quality of life.

Psoriasis is a chronic, inflammatory skin disease, characterized by scaling and inflammation, in which skin cells replicate at an extremely rapid rate. New skin cells are produced about eight times faster than normal—over several days instead of a month—but the rate at which old cells slough off is unchanged. About 80% of people with psoriasis suffer from the type of the disease known as “Psoriasis vulgaris.” It is also called plaque psoriasis because of the characteristic plaques on the skin: well-defined patches of red raised skin that can appear on any area of skin, although the knees, elbows, scalp, trunk and nails are the most common locations. The disease also may affect the fingernails, toenails, and the soft tissues inside the mouth and genitalia.

The flaky silvery white buildup on top of the plaques is called scale; it is composed of dead skin cells. This scale comes loose and sheds constantly from the plaques. Skin affected with psoriasis is generally very dry, and other possible symptoms include skin pain, itching and cracking. But psoriasis is more than cosmetic. The plaques may itch or burn. The skin at joints may crack. About 10 percent of people with psoriasis have joint inflammation that produces symptoms of arthritis. This condition is called psoriatic arthritis.

People with psoriasis may notice that there are times when their skin worsens, then improves. Conditions that may cause flare-ups include changes in climate, infections, stress, and dry skin.

Doctors usually diagnose psoriasis after a careful examination of the skin. However, diagnosis may be difficult because psoriasis often looks like other skin diseases. Sometimes a small piece of the skin is removed for a biopsy. A pathologist may assist with diagnosis by examining the small skin sample under a microscope.

There is no cure for psoriasis, but a broad range of treatments are available to reduce the symptoms, clear up the skin, and attempt to send the disease into remission. FDA-approved treatments range from creams rubbed into the skin, to lasers that aim ultraviolet rays at the skin, to the newest treatments—injectable drugs made from living cells. Over time, however, affected skin tends to resist some treatments. Also, a treatment that works in one person may have little effect in another. Thus, doctors commonly use a trial-and-error approach to finding a treatment that works, then switch treatments every 12 to 24 months to reduce resistance and adverse reactions. The purpose of the invention described herein is to overcome many of the shortcomings inherent in present treatment modalities.

### BRIEF DESCRIPTION OF THE DRAWINGS

FIG. 1 depicts a timeline used in the disclosed embodiments.

FIG. 2 depicts a graph showing increases in keratinocyte differentiation by sodium butyrate being inhibited by EGF.

FIG. 3 depicts a graph showing increases in keratinocyte differentiation by TCDD being inhibited by EGF.

FIG. 4 depicts a graph showing PD153035 increasing sodium butyrate mediated differentiation in the presence of EGF.

FIG. 5 depicts a graph showing PD153035 increasing TCDD-mediated differentiation in the presence of EGF.

### DETAILED DESCRIPTION OF THE OF THE INVENTION

This invention described herein is the combined use of inhibitors of the EGF signaling pathway and agents that promote cell differentiation in the treatment of chronic inflammatory skin diseases. This useful combination has never been conceived or described, nor is such a combination obvious to those with ordinary skill in the art.

EGF is a growth factor supporting the proliferation of epithelial cells. Numerous inhibitors of this pathway have been developed to inhibit or control the rate of proliferation of cells responding to this growth factor, including, but not limited to, psoriatic skin. Research leading to the present invention revealed that activators of the EGF receptor signaling pathway also prevent the ability of agents that promote differentiation to do so, leading to a less than effective treatment. Thus the method to combine agents that inhibit EGF receptor signaling and agents that promote cell differentiation should be useful in the treatment of diseases that involve cell proliferation, such as psoriasis and other chronic inflammatory skin diseases. In the presence of EGF receptor signaling, agents that promote differentiation are not effective. In the absence of an agent that can promote cell differentiation, inhibition of EGF receptor signaling alone is not effective in promoting cell differentiation. Thus, combining such agents will effectively inhibit cell proliferation and promote differentiation, resulting in treatment for chronic inflammatory skin disease that is more effective.

Inhibitors of the EGF receptor pathway would include, but not be limited to, agents that block the ligands of the EGF receptor from binding to the EGF receptor, agents that inhibit EGF receptor phosphorylation, such as PD153035. and agents that inhibit EGF receptor pathway signaling such as SU5271 and ZD1839.

The class of agents that promote cell differentiation are numerous and may include, but are not limited to the following compounds:
Ah receptor agonists, such as coal tar and polycyclic aromatic compounds;
heterocyclic compounds;
histone deacetylase inhibitors (HDAC inhibitors);
steroid hormones and analogs, including vitamin D and retinoic acid;
ultraviolet irradiation;
protein kinase C (PKC) activators;
lipids, including oxysterols and fatty acids
calcium and calcium ionphores
agents that increase intracellular calcium, for example thapsigargin
agents that increase intracellular cyclic AMP, for example forskolin
agents that activate Keap1/NRF2 signaling pathway, including dithiolethiones and oltipraz
naturally occurring products including bioactive polyphenols, flavonoids, alkaloids and terpinoids
chemotherapeutic agents including nucleosides, methotrexate;
psoralen ultraviolet A (PUVA) phototherapy.

In any specific embodiment, the medicament or pharmaceutical composition can be administered by any route suitable to each subject. Possible routes for administration include, but are not limited to topical, oral, injection, transdermal delivery, nasal spray, and suppository.

In one embodiment disclosed herein, the medicament is a topical pharmaceutical composition for the treatment of skin disorders. In this embodiment, the pharmaceutical composition comprises an efficacious concentration of an agent that promotes or accelerates cellular differentiation combined with an efficacious concentration of an agent that inhibits the EGF receptor signaling pathway; and a dermatologically acceptable carrier suitable for containment and delivery of the combined medicaments or pharmaceuticals to the skin. A dermatologically acceptable carrier is one that could be administered to the skin without undue toxicity, incompatibility, instability, allergic response, or otherwise result in degradation of the components or prevention their absorption, or adverse reactions. Dermatologically acceptable carriers may include petrolatum, mineral oil, ceresin, lanolin alcohol, or pantheon. Other dermatologically acceptable carriers are well known to persons with ordinary skill in the art, any of which may be used in a topical application of the pharmaceutical composition.

This topical form of the pharmaceutical composition is then administered directly to the affected area of the skin. In the alternative, the individual agents promoting differentiation and inhibiting the activation of the EGF receptor, each in a dermatologically acceptable carrier, may be administered sequentially to the skin within a period of 0-120 hours.

In one such embodiment for topical application, the differentiating agent, Calcipotriol at a concentration ranging from 0.0005-0.005% is combined with the EGF receptor signaling pathway inhibitor, PD153035 at a concentration ranging from 0.005-0.5% by weight (w/w). This would be combined in a dermatologically acceptable carrier. The combined agents would then be topically administered.

In another embodiment for topical application, the differentiating agent, Valproic acid at a concentration ranging from 0.01-10.0% could be combined with the EGF receptor signaling pathway inhibitor, PD153035 at a concentration ranging from 0.005-0.5% by weight (w/w). This would be combined in a dermatologically acceptable carrier. The combined agents would then be topically administered.

In yet another disclosed embodiment for topical application, the differentiating agent, suberolyanilide hydroxamic acid (SAHA) at a concentration ranging from 0.00001-0.01% is combined with the EGF receptor signaling pathway inhibitor, PD153035 at a concentration ranging from 0.005-0.5% by weight (w/w).

Another disclosed embodiment for topical application consists of the differentiating agent, Calcipotriol at a concentration ranging from 0.0005-0.005% combined with the EGF receptor signaling pathway inhibitor, SU5271 at a concentration ranging from 0.005-0.5% by weight (w/w).

Another disclosed embodiment of the pharmaceutical composition for topical application consists of the differentiating agent, Valproic acid at a concentration ranging from 0.01-10.0% combined with the EGF receptor signaling pathway inhibitor, SU5271 at a concentration ranging from 0.005-0.5% by weight (w/w).

A final disclosed embodiment for topical application consists of the differentiating agent, suberolyanilide hydroxamic acid (SAHA) at a concentration ranging from 0.00001-0.01% combined with the EGF receptor signaling pathway inhibitor, SU5271 at a concentration ranging from 0.005-0.5% by weight (w/w).

These disclosed embodiments for a topical treatment of chronic inflammatory skin diseases are just examples of combinations of EGF receptor inhibitors and agents that promote cell differentiation. Many different classes of EGF receptor inhibitors and cell differentiation agents are well known to persons skilled in the art, and can be used in the present invention.

As noted above, the medicament to be administered may also be an oral pharmaceutical composition. Such an embodiment for the treatment of skin disorders, would be comprised of the following:

an efficacious dose of an agent that promotes or accelerates cellular differentiation combined with an efficacious dose of an agent that inhibits the EGF receptor signaling pathway, and an acceptable carrier to deliver the medicaments as a pill or liquid.

The term “oral dose”, as used herein, means to take the medicament by mouth in order to deliver the dose systemically, in order to treat the skin disorder. The term “acceptable carrier” as used herein, means that the composition of the carrier are suitable for containment and delivery of the combined medicaments or pharmaceuticals by mouth, without undue toxicity, incompatibility, instability, allergic response and the like.

The term combined refers to either a combined set of pharmaceuticals in the same acceptable carrier, administered at the same time or, alternatively, a set of pharmaceuticals, each in an acceptable carrier, administered sequentially by mouth within a period of 0-120 hours.

One preferred embodiment for a oral dose consists of the differentiating agent, suberolyanilide hydroxamic acid (SAHA) at a dose ranging from 200-600 mg/day with the EGF receptor signaling pathway inhibitor, ZD1839 (lressa) at a dose of 250 or 500 mg/day.

The efficacy of the methods of treatment of skin disorders such as psoriasis described above was demonstrated in a study of primary cells in culture. In this study, Keratinocyte Cell Culture-Neonatal foreskin kerinocytes, purchased from Lonza (Walkersville, Md., USA), were grown in keratinocyte-SFM (Gilbro-BRL, Gaithersburg, Md., USA). This complete medium consists of keratinocyte basal medium and supplements (Gibco-BRL), human combinant EGF (5 ng/mL) and bovine pituitary extract (50 μg protein/mL). The calcium concentration in this medium is 0.09 mM.

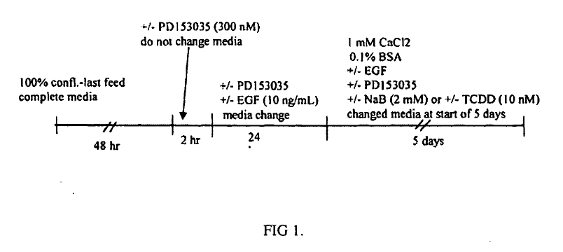

As can be seen in FIG. 1, cultures_were incubated at 37° C., 5% CO2, and the medium was replaced every two days. Unless indicated in the description for a particular figure set forth below, confluent fifth passage keratinocytes were used, with treatments beginning 2 days after the last addition of complete medium. The media was changed and the cells were pretreated with PD153035 (300 nM) for two hours. Media was again changed to contain PD153035 (300 nM) and epidermal growth factor (10 ng/mL) and left for 24 hours. The media was changed a final time to basal media supplemented with 1 mM CaCl2 and 0.1% bovine serum albumin with the indicated chemical additions. The cells were incubated at 37° C., 5% CO2 for 5 days before the comified envelopes were scored. The vehicle, DMSO, was used as the control for both PD153035 and TCDD.

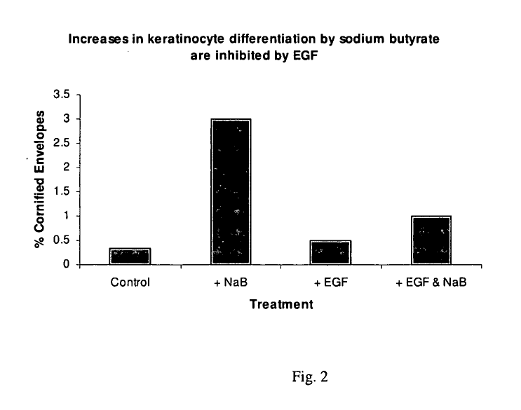

In reference to FIG. 2, Human keratinocytes were grown to confluence and treated with vehicle of media (control), sodium butyrate, epidermal growth factor (EGF) or a combination of EGF and NAB for 5 days. Cornified envelope formation is an endpoint of terminal differentiation. The percent of comified envelopes was calculated by dividing the number of cornified envelopes by the number of cells (times 100).

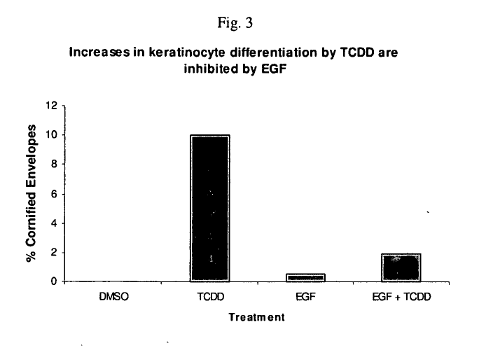

In FIG. 3, Human keratinocytes were grown to confluence and treated with vehicle of dimethylsulfoxide (DMSO), 2,3,7,8-tetrachlorodibenzo-p-dioxin (TCDD), epidermal growth factor (EFG) or a combination of EGF and TCDD for 5 days. Comified envelope formation is an endpoint of terminal cell differentiation. The percent of cornified envelopes was calculated by dividing the number of cornified envelopes by the number of cells (times 100).

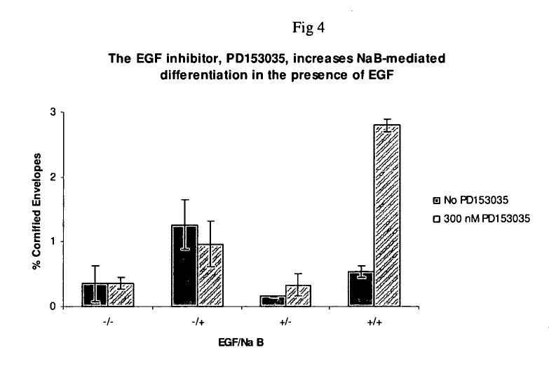

In FIG. 4, Human keratinocytes were grown to confluence and treated with vehicle of DMSO (−/−), sodium butyrate (NaB) alone (−/+); epidermal growth factor (EGF) alone (+/−) or a combination of EGF and NaB (+/+) with or without PD153035 for 5 days. Comified envelope formation is an endpoint of terminal cell differentiation. The average percent (n=3, +/−std dev) of cornified envelopes was calculated by dividing the number of cornified envelopes by the number of cells (times 100).

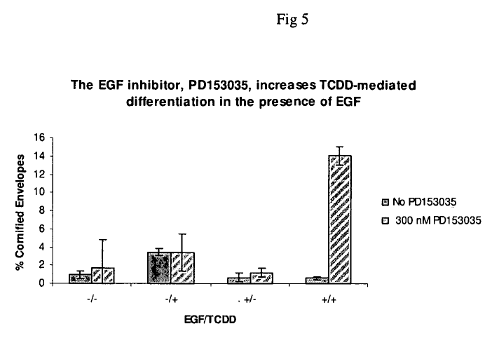

In FIG. 5, Human keratinocytes were grown to confluence and treated with vehicle of DMSO (−/−), TCDD alone (−/+), epidermal growth factor (EGF) alone (+/−) or a combination of EGF and TCDD (+/+) with or without PD153035 for 5 days. Cornified envelope formation is an endpoint of terminal cell differentiation. The average percent (n=3, +/−std dev) of comified envelopes was calculated by dividing the number of cornified envelopes by the number of cells (times 100).

As can be seen from the figures, it is clear that the combination of EGF inhibitor and differentiation promoter results in the greatest increase in terminal cell differentiation. The EFG inhibitor and differentiation promoter have a synergistic effect that results in a greater increase in cell differentiation than would be expected from their combination based on their individual results.

We claim:

1. A pharmaceutical composition for the treatment of chronic inflammatory skin disease comprising:
a. an inhibitor of the EGF receptor signaling pathway; and
b. an agent promoting cell differentiation.

2. The pharmaceutical composition of claim 1, wherein said inhibitor of the EGF receptor signaling pathway is selected from the list consisting of:
a. agents that block the binding of activating ligands of the EGF receptors;
b. agents that inhibit EGF receptor phosphorylation; and
c. agents that inhibit EGF receptor pathway signaling.

3. The pharmaceutical composition of claim 1, wherein said agent promoting cell differentiation is selected from the list consisting of:
a. Ah receptor agonists;
b. heterocyclic compounds;
c. histone deacetylase inhibitors;
d. steroid hormones;
e. analogs of steroid hormones;
f. ultraviolet irradiation;
g. psoralen ultraviolet A (PUVA) phototherapy
h. protein kinase C activators;
i. lipids including oxysterols, fatty acids and other;
j. calcium;
k. calcium ionphores;
l. agents that increase intracellular calcium;
m. agents that increase intracellular cyclic AMP;
n. agents that activate Keap1/NRF2 signaling pathway;
o. natural products; and
p. chemotherapeutic agents.

4. The pharmaceutical composition of claim 2, wherein said agent promoting cell differentiation is selected from the list comprising:
a. Ah receptor agonists;
b. heterocyclic compounds;
c. histone deacetylase inhibitors;
d. steroid hormones;
e. analogs of steroid hormones;
f. ultraviolet irradiation;
g. psoralen ultraviolet A (PUVA) phototherapy
h. protein kinase C activators;
i. lipids including oxysterols, fatty acids and other;
j. calcium;
k. calcium ionphores;
l. agents that increase intracellular calcium;
m. agents that increase intracellular cyclic AMP;
n. agents that activate Keap1/NRF2 signaling pathway;
o. natural products; and
p. chemotherapeutic agents.

5. The pharmaceutical composition of claim 1, further comprising a dermatologically acceptable carrier.

6. The pharmaceutical composition of claim 5, wherein said agent promoting cell differentiation is Calcipotriol, and wherein said inhibitor of the EGF signaling pathway is PD153035.

7. The pharmaceutical composition of claim 5, wherein said agent promoting cell differentiation is Valproic acid, and wherein said inhibitor of the EGF signaling pathway is PD153035.

8. The pharmaceutical composition of claim 5, wherein said agent promoting cell differentiation is suberolyanilide hydroxamic acid, and wherein said inhibitor of the EGF signaling pathway is PD153035.

9. The pharmaceutical composition of claim 5, wherein said agent promoting cell differentiation is Calcipotriol, and wherein said inhibitor of the EGF signaling pathway is SU5271.

10. The pharmaceutical composition of claim 5, wherein said agent promoting cell differentiation is Valproic acid, and wherein said inhibitor of the EGF signaling pathway is SU5271.

11. The pharmaceutical composition of claim 5, wherein said agent promoting cell differentiation is suberolyanilide hydroxamic acid, and wherein said inhibitor of the EGF signaling pathway is SU5271.

12. The pharmaceutical composition of claim 1, further comprising an orally acceptable carrier.

13. The pharmaceutical composition of claim 12, wherein said agent promoting cell differentiation is suberolyanilide hydroxamic acid, and wherein said inhibitor of the EGF signaling pathway is ZD1839.

14. A method for treating chronic inflammatory skin disease comprising administering the pharmaceutical composition of claim 1.

15. A method for treating chronic inflammatory skin disease sequentially over a period of 120 hours comprising:
a. administering an inhibitor of the EGF receptor signaling pathway; and
b. administering an agent promoting cell differentiation.
